(19) United States
(12) Patent Application Publication
(10) Pub. No.: US 2021/0220499 A1
(43) Pub. Date: Jul. 22, 2021

(54) ULTRAVIOLET IRRADIATION UNIT AND ULTRAVIOLET STERILIZER

(71) Applicant: Enplas Corporation, Saitama (JP)

(72) Inventor: Masato NAKAMURA, Saitama (JP)

(73) Assignee: Enplas Corporation

(21) Appl. No.: 17/226,934

(22) Filed: Apr. 9, 2021

Related U.S. Application Data
(63) Continuation of application No. PCT/JP2019/040134, filed on Oct. 10, 2019.

(30) Foreign Application Priority Data
Oct. 12, 2018 (JP) 2018-193673

(51) Int. Cl. A61L 2/10; C02F 1/32; B01D 53/00

(52) U.S. Cl. CPC A61L 2/10; C02F 1/325; B01D 53/007; B01D 2259/804; C02F 2303/04; C02F 2201/3228; C02F 2201/3227; A61L 2202/11

(57) ABSTRACT

An ultraviolet irradiation unit of the present invention includes a plurality of ultraviolet irradiation modules arranged and spaced apart from each other at intervals. Each of the ultraviolet irradiation modules includes a light source emitting ultraviolet rays, and a reflector arranged on an ultraviolet emission side of the light source and reflecting part of the ultraviolet rays emitted from the light source toward the object to be processed.

### CROSS-REFERENCE TO RELATED APPLICATIONS

This application is a Continuation Application of PCT Application No. PCT/JP2019/040134, filed Oct. 10, 2019 and based upon and claiming the benefit of priority from prior Japanese Patent Application No. 2018-193673, filed Oct. 12, 2018, the entire contents of all of which are incorporated herein by reference.

### BACKGROUND OF THE INVENTION

### 1. Field of the Invention

The present invention relates generally to an ultraviolet irradiation unit and an ultraviolet sterilizer.

### 2. Description of the Related Art

It is widely known that ultraviolet rays are used to sterilize a fluid in a flow channel tube. For example, according to JP 2016-531746 A, ultraviolet rays are uniformly dispersed to sterilize the fluid in the channel tube.

### BRIEF SUMMARY OF THE INVENTION

However, the fluid flowing in the flow channel tube is generally fast in the center of tube and slow on the tube wall side, in the laminar flow, and does not flow at a uniform velocity. Therefore, there is a risk that sterilization is nonuniformly performed and the fluid cannot be sufficiently sterilized in a conventional ultraviolet sterilizer.

One of embodiments described herein aims to provide an ultraviolet irradiation unit capable of sufficiently sterilizing a fluid evenly, and an ultraviolet sterilizer capable of sufficiently sterilizing a fluid flowing in a flow channel tube.

According to the present invention, there is provided an ultraviolet irradiation unit, which is an ultraviolet irradiation unit irradiating an object to be processed with ultraviolet rays for sterilization, and which includes a plurality of ultraviolet irradiation modules arranged and spaced apart from each other at intervals and emitting ultraviolet rays. Each of the ultraviolet irradiation modules includes a light source emitting ultraviolet rays, and a reflector arranged on an ultraviolet emission side of the light source and reflecting part of the ultraviolet rays emitted from the light source toward the object to be processed. The plurality of ultraviolet irradiation modules satisfy the following formula (1).

P/L(tan θ1+tan θ2)≤1 (1)

In formula (1), P referring to a distance (mm) between one ultraviolet irradiation module and the other ultraviolet irradiation module closest to the one ultraviolet irradiation module, L referring to an irradiation distance (mm) of the ultraviolet rays emitted from the one ultraviolet irradiation module and the other ultraviolet irradiation module, θ1 referring to a half value (°) of a half-width of a directivity angle of the ultraviolet rays emitted from the one ultraviolet irradiation module, θ2 referring to a half value (°) of a half-width of a directivity angle of the ultraviolet rays emitted from the other ultraviolet irradiation module.

According to the present invention, there is provided an ultraviolet sterilizer, which is an ultraviolet sterilizer irradiating an object to be processed flowing through a flow channel tube with ultraviolet rays for sterilization, and which includes a flow channel tube, and the above ultraviolet irradiation unit.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a cross-sectional view showing an ultraviolet irradiation unit according to an embodiment of the present invention.

FIG. 2 shows an ultraviolet irradiation module according to the embodiment, where (A) is a plan view, (B) is a bottom view, (C) is a side view, and (D) is a cross-sectional view taken along line D-D shown in (A).

FIG. 3 shows an ultraviolet irradiation unit according to the embodiment, where (A) is a plan view showing an ultraviolet irradiation unit comprising a plurality of ultraviolet irradiation modules arranged in a square lattice shape, and (B) is a plan view showing an ultraviolet irradiation unit comprising a plurality of ultraviolet irradiation modules arranged in a hexagonal lattice shape.

FIG. 4 is a diagram showing comparison while changing the distance between the ultraviolet irradiation modules, where (A1) to (C1) are plan views showing an ultraviolet irradiation unit comprising a plurality of ultraviolet irradiation modules arranged in a square lattice shape, (A2) to (C2) are distribution charts showing a ultraviolet irradiation intensity of the ultraviolet irradiation unit at an irradiation distance of 300 mm, and (A3) to (C3) are graphs showing the ultraviolet irradiation intensity of the ultraviolet irradiation unit at an irradiation distance of 300 mm.

FIG. 5 is a diagram showing comparison while changing the distance between the ultraviolet irradiation modules, where (A1) to (C1) are plan views showing an ultraviolet irradiation unit comprising a plurality of ultraviolet irradiation modules arranged in a hexagonal lattice shape, (A2) to (C2) are distribution charts showing a ultraviolet irradiation intensity of the ultraviolet irradiation unit at an irradiation distance of 300 mm, and (A3) to (C3) are graphs showing the ultraviolet irradiation intensity of the ultraviolet irradiation unit at an irradiation distance of 300 mm.

FIG. 6 is a diagram showing comparison while changing the distance between the ultraviolet irradiation modules, where (A1) to (C1) are plan views showing an ultraviolet irradiation unit comprising a plurality of ultraviolet irradiation modules arranged in a square lattice shape, (A2) to (C2) are distribution charts showing a ultraviolet irradiation intensity of the ultraviolet irradiation unit at an irradiation distance of 300 mm, and (A3) to (C3) are graphs showing the ultraviolet irradiation intensity of the ultraviolet irradiation unit at an irradiation distance of 300 mm.

FIG. 7 is a diagram showing comparison while changing the distance between the ultraviolet irradiation modules, where (A1) to (C1) are plan views showing an ultraviolet irradiation unit comprising a plurality of ultraviolet irradiation modules arranged in a hexagonal lattice shape, (A2) to (C2) are distribution charts showing a ultraviolet irradiation intensity of the ultraviolet irradiation unit at an irradiation distance of 300 mm, and (A3) to (C3) are graphs showing the ultraviolet irradiation intensity of the ultraviolet irradiation unit at an irradiation distance of 300 mm.

FIG. 8 is cross-sectional view showing an ultraviolet sterilizer according to the embodiment.

FIG. 9 is a diagram showing a first example of a reflector used in the embodiment, where (A) is a perspective view showing a reflector divisional part, and (B) is a perspective view showing a reflector in which the reflector divisional parts are combined.

FIG. 10 is a diagram showing a state of depositing aluminum on an ultraviolet reflection surface of the reflector by vapor deposition, where (A) is a cross-sectional view showing a state of depositing aluminum on an ultraviolet reflection surface of the reflector of the embodiment by vapor deposition, and (B) is a cross-sectional view showing a state of depositing aluminum on an ultraviolet reflection surface of the reflector of a first example by vapor deposition.

FIG. 11 is a diagram showing a second example of the reflector, where (A) is a perspective view showing a reflector divisional part, (B) is a plan view showing a state in which two reflector divisional parts are to be combined, and (C) is a perspective view showing the reflector formed by combining the reflector divisional parts.

FIG. 12 is a diagram showing a third example of the reflector, where (A) is a perspective view showing a reflector divisional part, (B) is a plan view showing a state in which four reflector divisional parts are to be combined, and (C) is a perspective view showing the reflector formed by combining the reflector divisional parts.

FIG. 13 is a diagram showing a fourth example of the reflector, where (A) is a perspective view showing a reflector divisional part, (B) is a plan view showing a state in which four reflector divisional parts are to be combined, and (C) is a perspective view showing the reflector formed by combining the reflector divisional parts.

FIG. 14 is a diagram showing a fifth example of the reflector, where (A) is a perspective view showing a reflector divisional part, and (B) is a perspective view showing a reflector in which the reflector divisional parts are combined.

FIG. 15 is a diagram showing a sixth example of the reflector, where (A) is a perspective view showing a reflector divisional part, (B) is a plan view showing a state in which two reflector divisional parts are to be combined, and (C) is a perspective view showing the reflector formed by combining the reflector divisional parts.

### DETAILED DESCRIPTION OF THE INVENTION

One of embodiments of the present invention will be described hereinafter with reference to the accompanying drawings.

### Embodiment

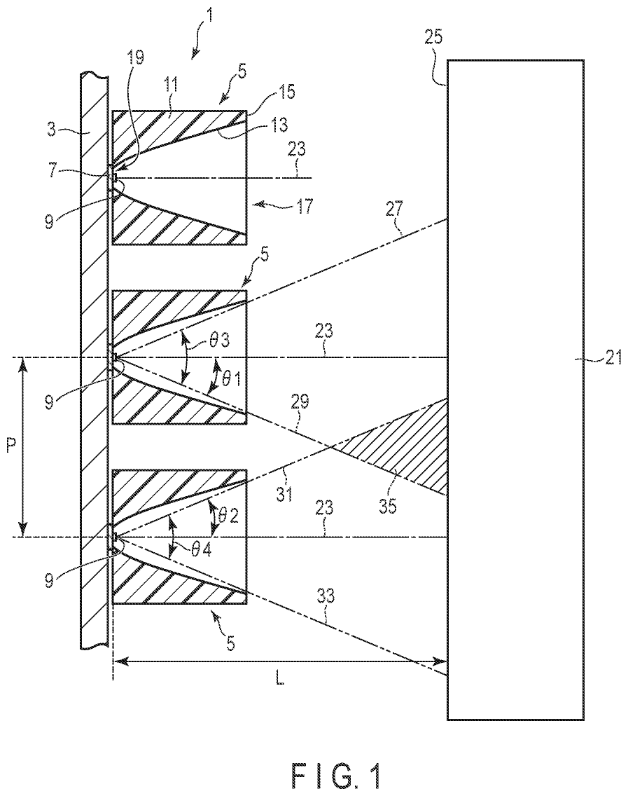

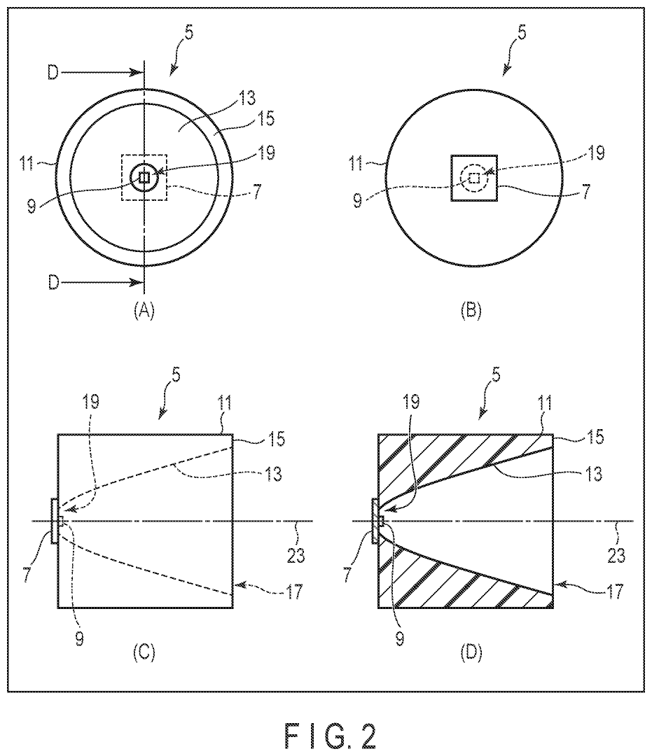

As shown in FIGS. 1 and 2, an ultraviolet irradiation unit 1 includes a base 3 and a plurality of ultraviolet irradiation modules 5 arranged on one surface of the base 3 and spaced at intervals. The ultraviolet irradiation module 5 includes a substrate 7 arranged on one surface of the base 3, a light source 9, and a reflector 11.

The light source 9 is arranged on one surface of the substrate 7. The light source 9 emits ultraviolet rays. The center wavelength or peak wavelength of the ultraviolet rays emitted from the light source 9 is, for example, 200 nm or more and 350 nm or less. The center wavelength or peak wavelength of the ultraviolet rays emitted from the light source 9 is desirably 260 nm or more and 290 nm or less from the viewpoint of high sterilization efficiency. The type of the light source 9 is not particularly limited as long as it can emit ultraviolet rays. The type of the light source 9 is, for example, a light emitting diode (LED), a mercury lamp, a metal halide lamp, a xenon lamp, or a laser diode (LD).

The reflector 11 is arranged on one surface of the substrate 7 so as to surround the light source 9. The reflector 11 comprises an ultraviolet reflection surface 13, an ultraviolet emission side surface 15, an ultraviolet emission side opening 17, and a substrate side opening 19. The reflector 11 reflects part of the ultraviolet rays emitted from the light source 9 (ultraviolet rays having a large emission angle) toward an object 21 to be processed arranged on the ultraviolet emission side. The reflector 11 is formed of, for example, a polycarbonate resin, an acrylic resin, or a cyclic olefin copolymer (COC). At least the surface of the ultraviolet reflecting surface 13 of the reflector 111 is coated with aluminum in a mirror shape. The surface of the ultraviolet emission side surface 15 may be coated to protect the resin from ultraviolet rays. The coating of the surfaces of the ultraviolet reflection surface 13 and the ultraviolet emission side surface 15 is, for example, aluminum coating by vapor deposition.

The ultraviolet reflection surface 13 reflects the ultraviolet rays emitted from the light source 9 and directly reaching the surface toward the object 21 to be processed. The ultraviolet reflection surface 13 is a rotation target surface with a central axis 23 serving as a rotation axis, and is circularly symmetric in the present embodiment, and is shaped in a concave curve with respect to the central axis 23. The ultraviolet reflection surface 13 may be shaped in a convex curve or shaped linearly in the direction of the central axis 23, such that a purpose of deflecting the ultraviolet rays emitted from the light source 9 toward the object 21 to be processed can be achieved. If the surface of the ultraviolet emission side surface 15 is coated with the same coating as that of the ultraviolet reflection surface 13, part of the ultraviolet rays that reach the object 21 to be processed and are reflected can be reflected toward the object 21 to be processed by the ultraviolet emission side surface 15.

The ultraviolet emission side opening 17 is larger than the substrate side opening 19. The ultraviolet emission side opening 17 has, for example, a circular shape with a diameter of 15 mm. The substrate side opening 19 has, for example, a circular shape with a diameter of 2.8 mm. The light source 9 is arranged in the center of the substrate side opening 19.

The object 21 to be processed is, for example, a gas such as air, a grain such as wheat flour or other powder, a fluid such as liquid such as tap water or agricultural water.

The plurality of ultraviolet irradiation modules 5 including the light sources 9 and the reflectors 11 satisfy the following formula (1):

P/L(tan θ1+tan θ2)≤1 (1)

As shown in FIG. 1, in the above formula (1), P refers to a distance (mm) between one ultraviolet irradiation module 5 and the other ultraviolet irradiation module 5 closest to the ultraviolet irradiation module 5. More specifically, P refers to the distance (mm) between the light source 9 of one ultraviolet irradiation module 5 and the light source 9 of the other ultraviolet irradiation module 5. P is, for example, 15 mm to 50 mm.

In the above formula (1), L refers to the irradiation distance (mm) of the ultraviolet rays emitted from one and the other ultraviolet irradiation modules 5. More specifically, L refers to the distance (mm) from the light source 9 to a surface 25 to be irradiated of the object 21 to be processed. L is, for example, 20 mm to 1,000 mm.

In the above formula (1), θ1 is a half value (°) of the half-width of the directivity angle of the ultraviolet rays emitted from one ultraviolet irradiation module 5. The half width of directivity angle of the ultraviolet rays is the angle between the directions in which the intensity of ultraviolet rays is half the maximum value. More specifically, the half width of directivity angle of the ultraviolet rays emitted from one ultraviolet irradiation module 5 is an angle θ3 between an axis 27 and an axis 29. θ1 is the angle between the central axis 23 and the axis 29, and is also half the value of θ3. θ1 is, for example, 0.1° to 30.0°.

In the above formula (1), θ2 is a half value (°) of the half width of directivity angle of the ultraviolet rays emitted from the other ultraviolet irradiation module 5. More specifically, the half width of directivity angle of the ultraviolet rays emitted from the other ultraviolet irradiation module 5 is an angle θ4 between an axis 31 and an axis 33. θ2 is the angle between the central axis 23 and the axis 31, and is also half the value of θ4. θ2 is, for example, 0.1° to 30.0°.

When θ1 and θ2 are the same in the above formula (1), the plurality of ultraviolet irradiation modules 5 satisfy the following formula (2):

P/L tan θ3≤2 (2)

In the above formula (2), P and L are the same as those defined in formula (1), and θ3 has the same definition as θ1 or θ2 defined in formula (1). θ3 is, for example, 0.1° to 30.0°, similarly to θ1 and θ2.

As shown in FIG. 1, the ultraviolet irradiation module 5 satisfying the above formula (1) generates an ultraviolet overlapping part 35 where part of the ultraviolet rays emitted from one ultraviolet irradiation module 5 overlaps part of the ultraviolet rays emitted from the other ultraviolet irradiation module 5 on the surface 25 to be irradiated of the object 21 to be processed. The ultraviolet overlapping part 35 has an ultraviolet irradiation intensity higher than that at a part where the ultraviolet rays do not overlap, and has an ultraviolet irradiation intensity capable of sufficiently sterilizing the object 21 to be processed. For this reason, the ultraviolet radiation module 5 satisfying the above formula (1) can sufficiently sterilize the object 21 to be processed by generating the ultraviolet overlapping part 35.

In contrast, the ultraviolet radiation module which does not satisfy the above formula (1) cannot generate an ultraviolet overlapping part or, even if an ultraviolet overlapping part is generated, the ultraviolet overlapping part is very small. In addition, the ultraviolet irradiation module which does not satisfy the above formula (1) generates a region which the ultraviolet rays do not reach on the surface to be irradiated of the object to be processed, and tends to generate unevenness of ultraviolet rays. For this reason, the ultraviolet irradiation module 5 which does not satisfy the above formula (1) cannot emit ultraviolet rays having high ultraviolet radiation intensity, and tends to cause unevenness in ultraviolet rays, and thus, there is a risk that the object to be processed cannot be sufficiently sterilized.

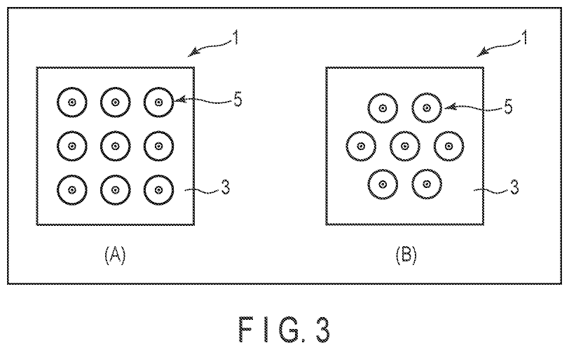

As shown in (A) of FIG. 3, the ultraviolet irradiation unit 1 may comprise a plurality of ultraviolet irradiation modules 5 arranged in a square lattice shape in planar view. As shown in (B) of FIG. 3, the ultraviolet irradiation unit 1 may comprise a plurality of ultraviolet irradiation modules 5 arranged in a hexagonal lattice shape in planar view. The ultraviolet irradiation unit 1 comprises, for example, seven to sixteen ultraviolet irradiation modules 5. In addition, the ultraviolet irradiation unit 1 may be arranged in an oblique lattice shape or a rectangular lattice shape in planar view.

The ultraviolet irradiation unit 1 shown in (A) and (B) of FIG. 3 can generate the ultraviolet overlapping part having a high ultraviolet irradiation intensity at a position near the center of the emitted ultraviolet rays. For this reason, such an ultraviolet irradiation unit 1 irradiates a fluid flowing through the flow channel tube, i.e., the fluid of a laminar flow which is fast at the center of the tube and slow on the tube wall side, with ultraviolet rays having an ultraviolet overlapping part having a high ultraviolet irradiation intensity in the vicinity of the center having a high speed, from a position facing the fluid flow, and can thereby sufficiently sterilize the fluid.

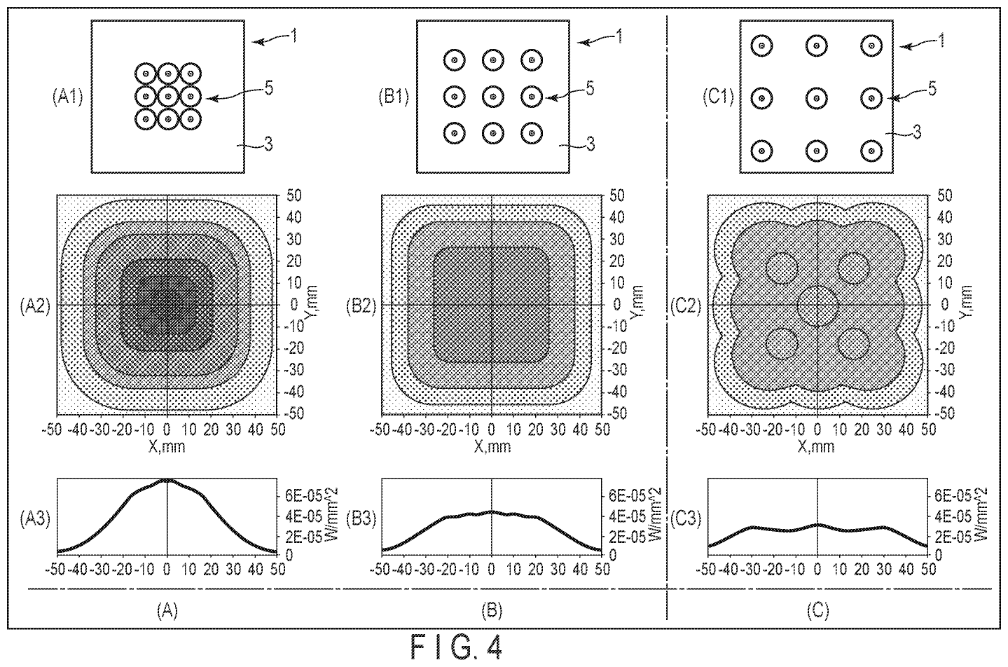

The ultraviolet irradiation intensity of the ultraviolet irradiation unit 1 comprising the plurality of ultraviolet irradiation modules 5 arranged in a square lattice shape or a hexagonal lattice shape in planar view will be described below with reference to FIG. 4 to FIG. 7.

The ultraviolet irradiation unit 1 shown in (A1) to (C1) of FIG. 4 has the same configuration except that the distance between the ultraviolet irradiation modules 5 is different. In each ultraviolet irradiation unit 1 shown in (A1) to (C1) of FIG. 4, θ3 is 2.8° and L is 300 mm in the above formula (2).

The distance P between the ultraviolet irradiation modules 5 shown in (A1) of FIG. 4 is 15.0 mm. Therefore, in the ultraviolet irradiation unit 1 shown in (A1) of FIG. 4,

P/L tan θ3=1.02<2

The distance P between the ultraviolet irradiation modules 5 shown in (B1) of FIG. 4 is 22.5 mm. Therefore, in the ultraviolet irradiation unit 1 shown in (B1) of FIG. 4,

P/L tan θ3=1.53<2

The distance P between the ultraviolet irradiation modules 5 shown in (C1) of FIG. 4 is 30.0 mm. Therefore, in the ultraviolet irradiation unit 1 shown in (C1) of FIG. 4,

P/L tan θ3=2.04>2

As shown in (A2), (A3), (B2), and (B3) of FIG. 4, it can be recognized that the ultraviolet irradiation unit 1 satisfying the above formula (2), i.e., (1), generates ultraviolet rays including an ultraviolet overlapping part having a high ultraviolet irradiation intensity in the vicinity of the center.

In contrast, as shown in (C2) and (C3) of FIG. 4, the ultraviolet irradiation unit 1 that does not satisfy the above formula (2), i.e., (1) cannot irradiate the ultraviolet rays having a high ultraviolet irradiation intensity in the vicinity of the center.

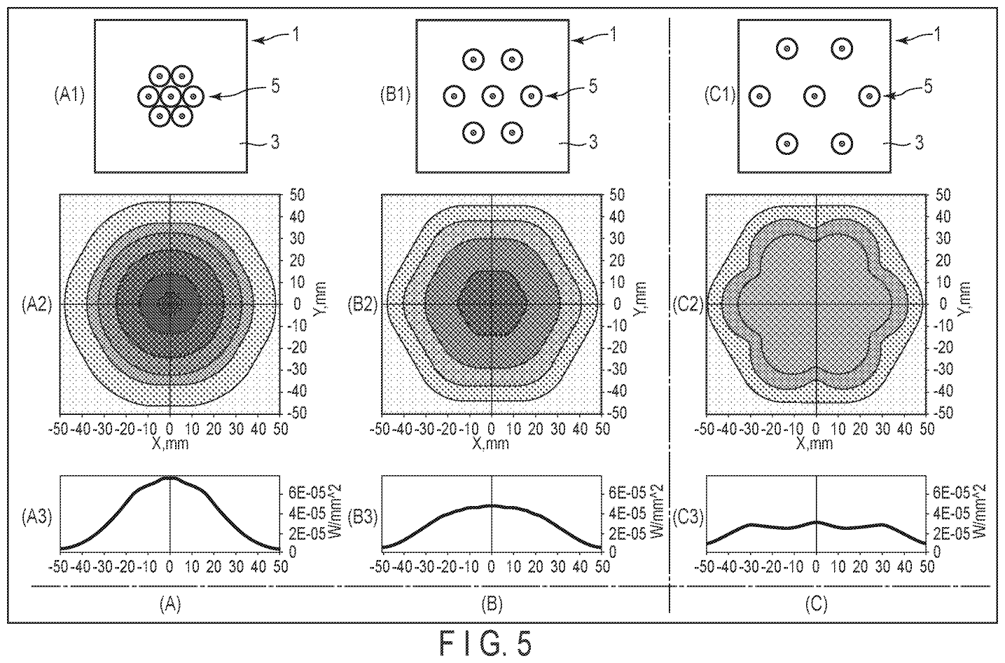

The ultraviolet irradiation unit 1 shown in (A1) to (C1) of FIG. 5 has the same configuration except that the distance between the ultraviolet irradiation modules 5 is different. In each ultraviolet irradiation unit 1 shown in (A1) to (C1) of FIG. 5, θ3 is 2.8° and L is 300 mm in the above formula (2).

The distance P between the ultraviolet irradiation modules 5 shown in (A1) of FIG. 5 is 15.0 mm. Therefore, in the ultraviolet irradiation unit 1 shown in (A1) of FIG. 5,

P/L tan θ3=1.02<2

The distance P between the ultraviolet irradiation modules 5 shown in (B1) of FIG. 5 is 22.5 mm. Therefore, in the ultraviolet irradiation unit 1 shown in (B1) of FIG. 5,

P/L tan θ3=1.53<2

The distance P between the ultraviolet irradiation modules 5 shown in (C1) of FIG. 5 is 30.0 mm. Therefore, in the ultraviolet irradiation unit 1 shown in (C1) of FIG. 5,

### P/L tan θ3=2.04>2

As shown in (A2), (A3), (B2), and (B3) of FIG. 5, it can be recognized that the ultraviolet irradiation unit 1 satisfying the above formula (2), i.e., (1), generates ultraviolet rays including an ultraviolet overlapping part having a high ultraviolet irradiation intensity in the vicinity of the center.

In contrast, as shown in (C2) and (C3) of FIG. 5, the ultraviolet irradiation unit 1 that does not satisfy the above formula (2), i.e., (1) cannot irradiate the ultraviolet rays having a high ultraviolet irradiation intensity in the vicinity of the center.

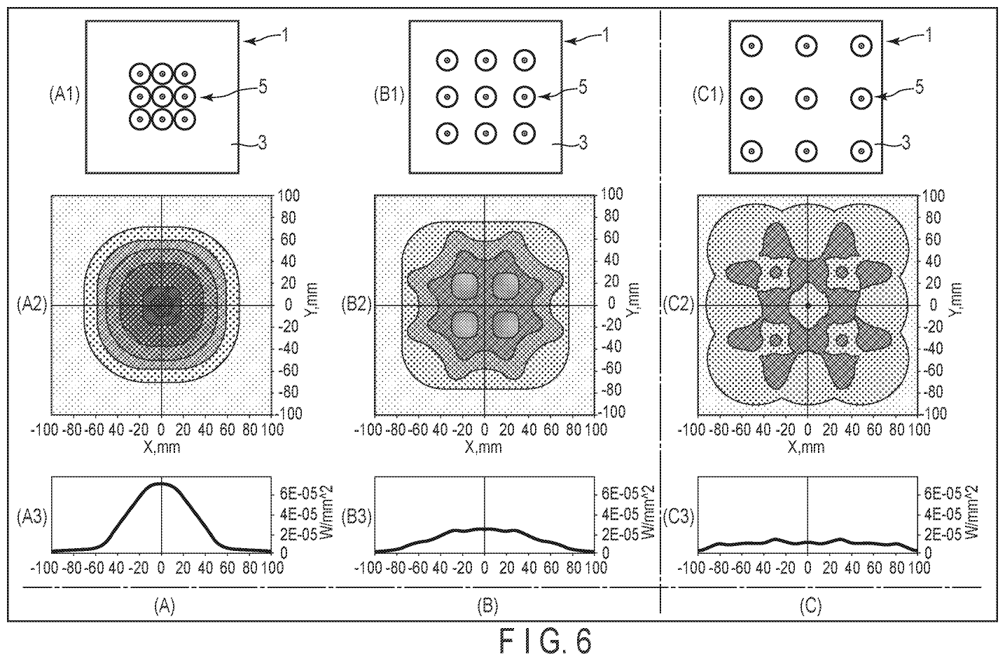

The ultraviolet irradiation unit 1 shown in (A1) to (C1) of FIG. 6 has the same configuration except that the distance between the ultraviolet irradiation modules 5 is different. In each ultraviolet irradiation unit 1 shown in (A1) to (C1) of FIG. 6, θ3 is 5.7° and L is 300 mm in the above formula (2).

The distance P between the ultraviolet irradiation modules 5 shown in (A1) of FIG. 6 is 15.0 mm. Therefore, in the ultraviolet irradiation unit 1 shown in (A1) of FIG. 6,

P/L tan θ3=0.50<2

The distance P between the ultraviolet irradiation modules 5 shown in (B1) of FIG. 6 is 37.5 mm. Therefore, in the ultraviolet irradiation unit 1 shown in (B1) of FIG. 6,

P/L tan θ3=1.25<2

The distance P between the ultraviolet irradiation modules 5 shown in (C1) of FIG. 6 is 60.0 mm. Therefore, in the ultraviolet irradiation unit 1 shown in (C1) of FIG. 6,

P/L tan θ3=2.01>2

As shown in (A2), (A3), (B2), and (B3) of FIG. 6, it can be recognized that the ultraviolet irradiation unit 1 satisfying the above formula (2), i.e., (1), generates ultraviolet rays including an ultraviolet overlapping part having a high ultraviolet irradiation intensity in the vicinity of the center.

In contrast, as shown in (C2) and (C3) of FIG. 6, the ultraviolet irradiation unit 1 that does not satisfy the above formula (2), i.e., (1) cannot irradiate the ultraviolet rays having a high ultraviolet irradiation intensity in the vicinity of the center.

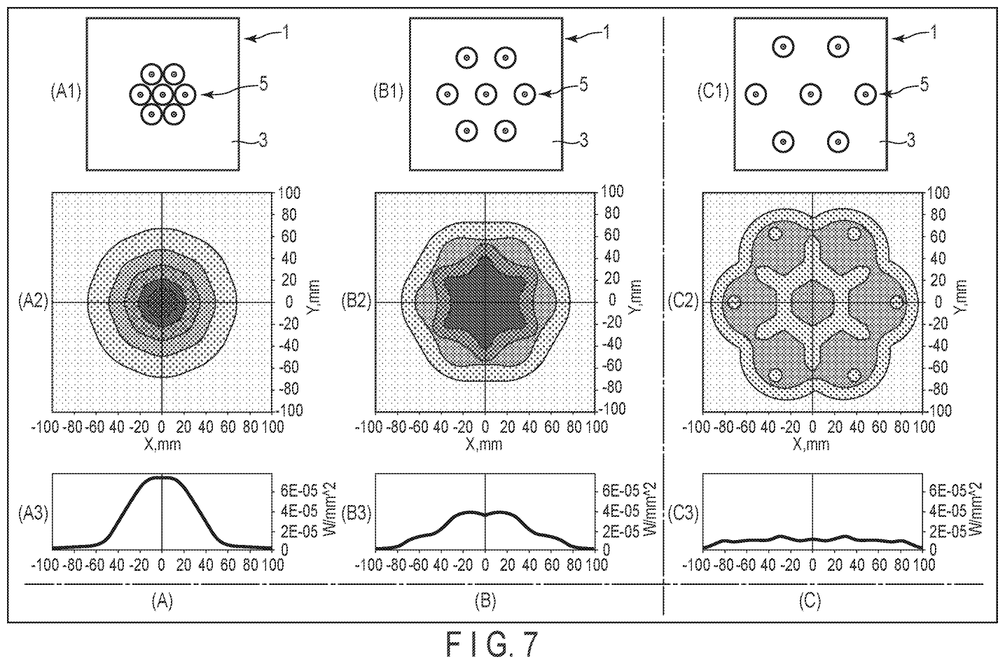

The ultraviolet irradiation unit 1 shown in (A1) to (C1) of FIG. 7 has the same configuration except that the distance between the ultraviolet irradiation modules 5 is different. In each ultraviolet irradiation unit 1 shown in (A1) to (C1) of FIG. 7, θ3 is 5.7° and L is 300 mm in the above formula (2).

The distance P between the ultraviolet irradiation modules 5 shown in (A1) of FIG. 7 is 15.0 mm. Therefore, in the ultraviolet irradiation unit 1 shown in (A1) of FIG. 7,

P/L tan θ3=0.50<2

The distance P between the ultraviolet irradiation modules 5 shown in (B1) of FIG. 7 is 37.5 mm. Therefore, in the ultraviolet irradiation unit 1 shown in (B1) of FIG. 7,

P/L tan θ3=1.25<2

The distance P between the ultraviolet irradiation modules 5 shown in (C1) of FIG. 7 is 60.0 mm. Therefore, in the ultraviolet irradiation unit 1 shown in (C1) of FIG. 7,

P/L tan θ3=2.01>2

As shown in (A2), (A3), (B2), and (B3) of FIG. 7, it can be recognized that the ultraviolet irradiation unit 1 satisfying the above formula (2), i.e., (1), generates ultraviolet rays including an ultraviolet overlapping part having a high ultraviolet irradiation intensity in the vicinity of the center.

In contrast, as shown in (C2) and (C3) of FIG. 7, the ultraviolet irradiation unit 1 that does not satisfy the above formula (2), i.e., (1) cannot emit ultraviolet rays having a high ultraviolet irradiation intensity in the vicinity of the center.

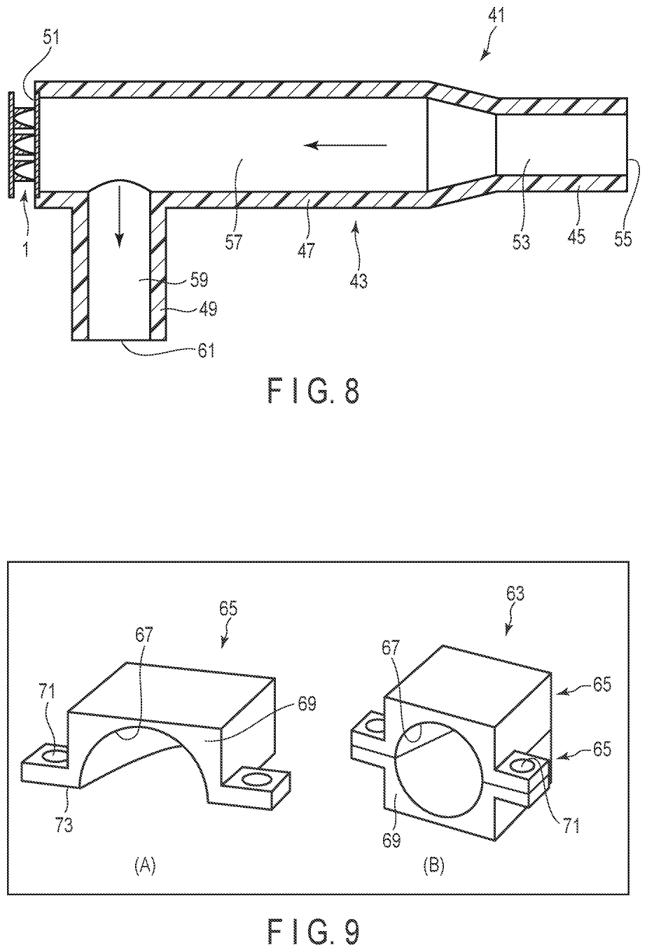

As shown in FIG. 8, an ultraviolet sterilizer 41 includes the ultraviolet irradiation unit 1 and a flow channel tube 43.

The flow channel tube 43 includes an inflow part 45, a flow channel tube body 47, an outflow part 49, and an ultraviolet incidence window 51. The flow channel tube 43 is a tube through which the fluid to be sterilized flows. The flow channel tube 43 is formed of a material such as polypropylene (PP), polytetrafluoroethylene (PTFE), stainless steel, or aluminum, which can hardly be deformed or damaged by the pressure of the fluid.

The inflow part 45 includes an inflow channel 53 inside. An upstream end of the inflow part 45 is an inflow port 55 for allowing the fluid to flow into the inflow channel 53. A downstream end of the inflow part 45 is connected to the tube wall of the upstream end of the flow channel tube body 47. The inflow part 45 is connected to a fluid supply device or the like (not shown) via the inflow port 55, and guides the fluid from the fluid supply device to the flow channel tube body 47. The inflow port 55 may have a shape in which a hose for guiding the fluid to the inflow channel 53 can be fitted. The inflow part 45 introduces the fluid sterilized by irradiation of ultraviolet rays into the flow channel tube body 47.

The flow channel tube body 47 includes a processing flow channel 57 that flows from one end side toward the other end side. The shape of the flow channel tube body 47 is not particularly limited as long as the fluid can flow. The shape of the processing flow channel 57 may be linear or curved. In the present embodiment, the processing flow channel 57 has a linear shape. In addition, the cross-sectional shape of the processing flow channel 57 in the direction perpendicular to the direction in which the fluid flows is not particularly limited either. The cross-sectional shape may be circular or polygonal. In the present embodiment, the cross-sectional shape of the processing flow channel 57 in the direction perpendicular to the direction in which the fluid flows is circular. The flow channel tube body 47 may be of any size that enables the fluid to be sufficiently sterilized by irradiation of ultraviolet rays. The flow channel length of the flow channel tube body 47 can be set to, for example, 2 cm or more and 100 cm or less.

The outflow part 49 includes an outflow channel 59 inside. The upstream end of the outflow part 49 is connected to the vicinity of the downstream end of the flow channel tube body 47. The downstream end of the outflow part 49 is an outflow port 61 for guiding to a liquid storage device or the like (not shown). The outflow port 61 is connected to the liquid storage device and guides the fluid from the processing flow channel 57 to the liquid storage device or the like. The outflow port 61 may have a shape into which a hose for guiding the fluid to the liquid storage device can be fitted. The outflow part 49 causes the sterilized fluid to flow out from the processing flow channel 57.

The ultraviolet incidence window 51 is fitted into an end opening on the downstream side of the flow channel tube body 47. The ultraviolet incidence window 51 allows the ultraviolet rays emitted from the ultraviolet irradiation unit 1 to pass through the flow channel tube 43 (flow channel tube body 47). The ultraviolet incidence window 51 is formed of a material having a high transmittance for ultraviolet rays, for example, quartz (SiO₂), sapphire (Al₂O₃), or an amorphous fluorine resin.

The fluid introduced from the inflow port 55 into the processing flow channel 57 via the inflow channel 53 is irradiated with the ultraviolet rays emitted from the ultraviolet irradiation unit 1 while flowing through the processing flow channel 57 and is sterilized. The irradiation direction of the ultraviolet rays emitted from the ultraviolet irradiation unit 1 is desirably opposed to the fluid flow. After that, the sterilized fluid is discharged from the outflow port 61 via the outflow channel 59.

The flow rate of the fluid may be sufficiently fast to be sufficiently sterilized by the irradiation of ultraviolet rays while it flows through the processing flow channel 57, and is, for example, equal to or less than 5 L/min to 100 L/min.

Since such an ultraviolet sterilizer 41 comprises the above-described ultraviolet irradiation unit 1, the sterilizer can sufficiently sterilize the fluid passing through the flow channel tube, i.e., the fluid of a laminar flow which is fast at the center of the tube and slow on the tube wall side, by irradiating the fluid with ultraviolet rays having an ultraviolet overlapping part having a high ultraviolet irradiation intensity in the vicinity of the center, from a position opposed to the fluid flow.

As shown in FIG. 9, a reflector 63 of a first example is composed of two reflector divisional parts 65. The reflector dividing part 65 includes an ultraviolet reflection surface 67, an ultraviolet emission side surface 69, a screw hole 71, and an engaging surface 73. In the reflector 63, the engaging surface 73 of one reflector divisional part 65 and the engaging surface 73 of the other reflector divisional part 65 are aligned with each other and, for example, a screw (not shown) is inserted into the screw hole 71 and is fixed by a nut (not shown).

By dividing and manufacturing the reflector similarly to the reflector 63 of the first example, the direction of the ultraviolet reflection surface 67 can be arbitrarily set when the aluminum coating is performed on the ultraviolet reflection surface 67 by vapor deposition.

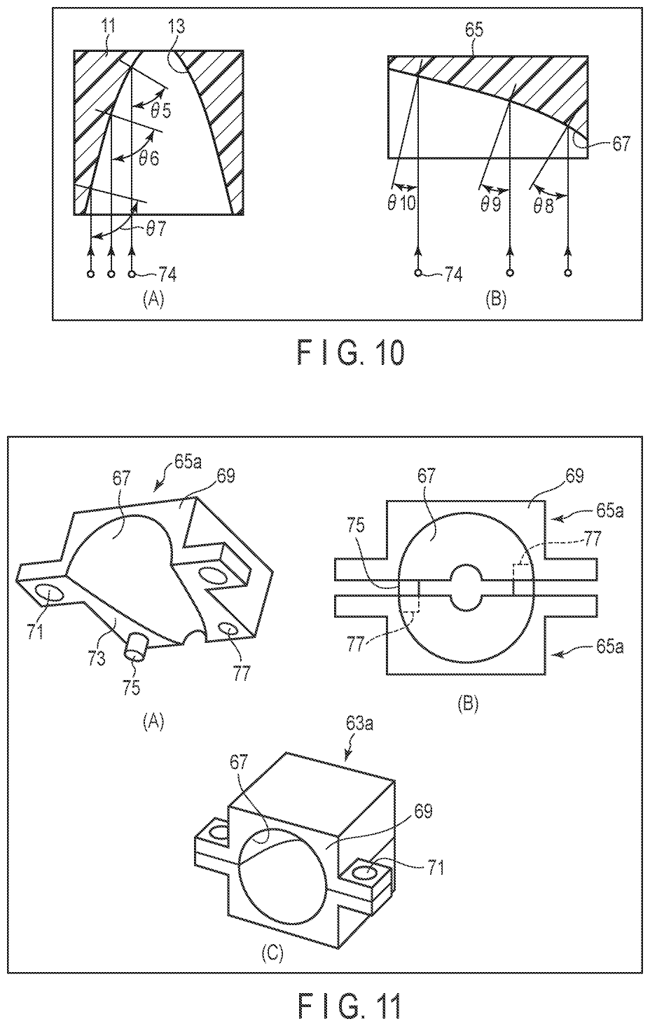

That is, as shown in (A) and (B) of FIG. 10, angles θ8, θ9, and θ10 formed by the direction in which the aluminum vapor deposition material 74 faces and the normal of the ultraviolet reflection surface 13, when the ultraviolet reflection surfaces 67 of the reflector divisional parts 65 are coated with aluminum by vapor deposition in the first example, can be made smaller than angles θ5, θ6, and θ7 formed by the direction in which the aluminum vapor deposition material 74 faces and the normal of the ultraviolet reflection surface 13, when the ultraviolet reflection surface 13 of the reflector 11 is coated with aluminum by vapor deposition in the embodiment. For this reason, aluminum can easily be deposited to a desired thickness by vapor deposition, on the ultraviolet reflection surfaces 67 of the reflector dividing parts 65. Therefore, by the reflector divisional parts 65, a desired reflectance can be given to the ultraviolet reflection surface 67.

A reflector 63a of a second example will be described with reference to FIG. 11. A reflector 63a of a second example is different from the reflector 63 of the first example with respect to the only structure of reflector divisional parts 65a. The same constituent elements of the reflector 63a of the second example as those of the reflector 63 of the first example will be denoted by the same reference numerals and detailed descriptions will be omitted.

The reflector 63a of the second example includes two reflector divisional parts 65a. The reflector divisional part 65a includes a positioning pin 75 and a positioning hole 77. In the reflector 63a, the positioning pin 75 of one reflector divisional part 65a is inserted into the positioning hole 77 of the other reflector divisional part 65a and, subsequently, the engaging surface 73 of one reflector divisional part 65a and the engaging surface 73 of the other reflector divisional part 65a are aligned with each other.

Since such a reflector 63a of the second example has the same effects as those of the reflector 63 of the first example and since the positioning pin 75 of one reflector divisional part 65a is inserted into the positioning hole 77 of the other reflector divisional part 65a, the reflector 63a can easily be formed without misalignment.

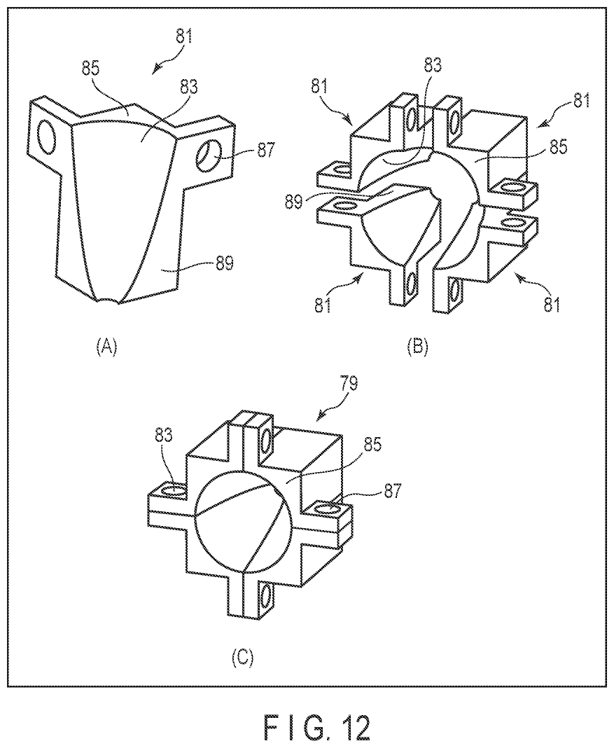

As shown in FIG. 12, a reflector 79 of a third example is composed of four reflector divisional parts 81. The reflector dividing part 81 includes an ultraviolet reflection surface 83, an ultraviolet emission side surface 85, a screw hole 87, and an engaging surface 89. In the reflector 79, the engaging surface 89 of one reflector divisional part 81 and the engaging surface 89 of the other reflector divisional part 81 are aligned with each other and, for example, a screw (not shown) is inserted into the screw hole 87 and is fixed by a nut (not shown).

The reflector 79 of the third example has the same advantages as the reflector 63 of the first example.

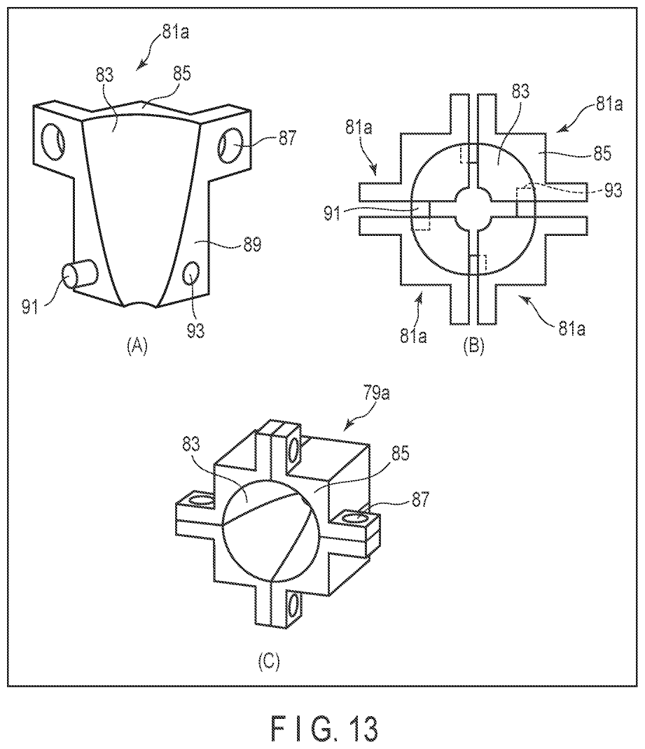

A reflector 79a of a fourth example will be described with reference to FIG. 13. A reflector 79a of a fourth example is different from the reflector 79 of the third example with respect to the only structure of reflector divisional parts 81a. The same constituent elements of the reflector 79a and the reflector divisional parts 81a of the fourth example as those of the reflector 79 of the third example will be denoted by the same reference numerals and detailed descriptions will be omitted.

The reflector 79a of the fourth example includes four reflector divisional parts 81a. The reflector divisional part 81a includes a positioning pin 91 and a positioning hole 93. In the reflector 79a, the positioning pin 91 of one reflector divisional part 81a is inserted into the positioning hole 93 of the other reflector divisional part 81a and, subsequently, the engaging surface 89 of one reflector divisional part 81a and the engaging surface 89 of the other reflector divisional part 81a are aligned with each other.

Since such a reflector 79a of the fourth example has the same effects as those of the reflector 79 of the third example and since the positioning pin 91 of one reflector divisional part 81a is inserted into the positioning hole 93 of the other reflector divisional part 81a, the reflector 79a can easily be formed without misalignment.

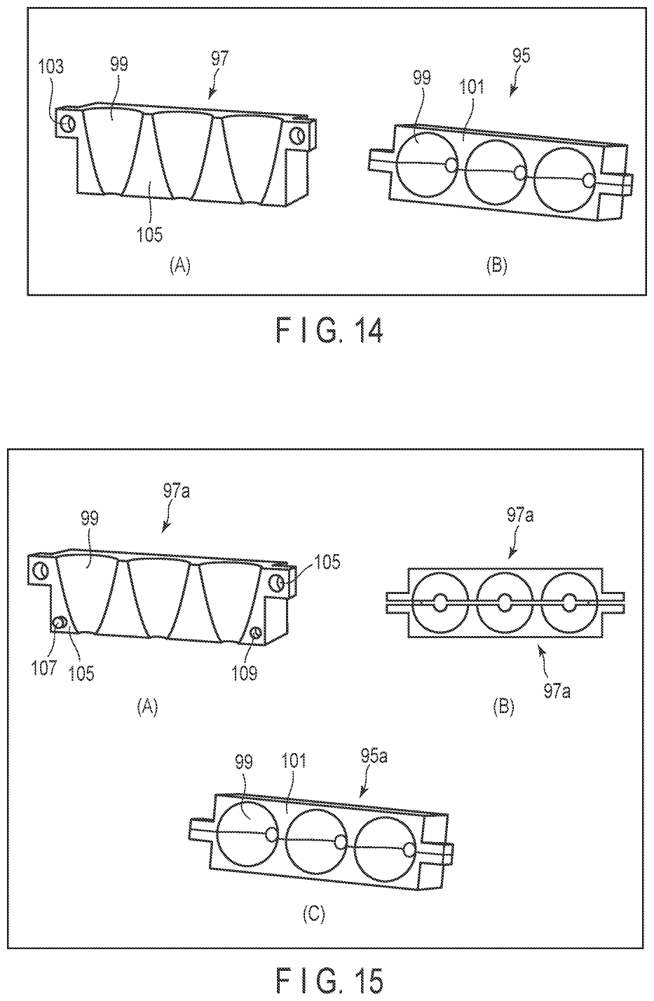

As shown in FIG. 14, a reflector 95 of a fifth example is formed by integrating three reflector parts and composed of two reflector divisional parts 97. The reflector dividing part 97 includes an ultraviolet reflection surface 99, an ultraviolet emission side surface 101, a screw hole 103, and an engaging surface 105. In the reflector 95, the engaging surface 105 of one reflector divisional part 97 and the engaging surface 105 of the other reflector divisional part 97 are aligned with each other and, for example, a screw (not shown) is inserted into the screw hole 103 and is fixed by a nut (not shown).

The reflector 95 of the fifth example has the same advantages as the reflector 63 of the first example and can simultaneously form three reflector parts.

A reflector 95a of a sixth example will be described with reference to FIG. 15. A reflector 95a of a sixth example is different from the reflector 95 of the fifth example with respect to the only structure of reflector divisional parts 97a. The same constituent elements of the reflector 95a and the reflector divisional parts 97a of the sixth example as those of the reflector 95 of the fifth example will be denoted by the same reference numerals and detailed descriptions will be omitted.

The reflector 95a of the sixth example includes two reflector divisional parts 97a. The reflector divisional part 97a includes a positioning pin 107 and a positioning hole 109. In the reflector 95a, the positioning pin 107 of one reflector divisional part 97a is inserted into the positioning hole 109 of the other reflector divisional part 97a and, subsequently, the engaging surface 105 of one reflector divisional part 97a and the engaging surface 105 of the other reflector divisional part 97a are aligned with each other.

Since such a reflector 95a of the sixth example has the same effects as those of the reflector 95 of the fifth example and since the positioning pin 107 of one reflector divisional part 97a is inserted into the positioning hole 109 of the other reflector divisional part 97a, the reflector can easily be formed without misalignment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultraviolet irradiation unit irradiating an object to be processed with ultraviolet rays for sterilization, including a plurality of ultraviolet irradiation modules arranged and spaced apart from each other at intervals and emitting ultraviolet rays,
each of the ultraviolet irradiation modules comprising:
a light source emitting ultraviolet rays; and
a reflector arranged on an ultraviolet emission side of the light source and reflecting a part of the ultraviolet rays emitted from the light source toward the object to be processed,
the plurality of ultraviolet irradiation modules satisfying formula (1)
P/L (tan θ1+tan θ2)≤1 (1)
in formula (1), P referring to a distance (mm) between one ultraviolet irradiation module and the other ultraviolet irradiation module closest to the one ultraviolet irradiation module, L referring to an irradiation distance (mm) of the ultraviolet rays emitted from the one ultraviolet irradiation module and the other ultraviolet irradiation module, θ1 referring to a half value (°) of a half width of a directivity angle of the ultraviolet rays emitted from the one ultraviolet irradiation module, θ2 referring to a half value (°) of a half width of a directivity angle of the ultraviolet rays emitted from the other ultraviolet irradiation module.

2. The ultraviolet irradiation unit of claim 1, wherein
the plurality of ultraviolet irradiation modules are arranged in a square lattice shape or a hexagonal lattice shape in planar view.

3. The ultraviolet irradiation unit of claim 1, wherein
the reflector is formed by combining a plurality of reflector divisional parts.

4. An ultraviolet sterilizer irradiating a fluid flowing through a flow channel tube with ultraviolet rays for sterilization,
comprising a flow channel tube and the ultraviolet irradiation unit of claim 1.

5. The ultraviolet sterilizer of claim 4, wherein
the ultraviolet irradiation unit is arranged in the flow channel tube to make an irradiation direction of ultraviolet rays opposed to a flow of the fluid.
